(19) United States
(12) Patent Application Publication
(10) Pub. No.: US 2021/0065026 A1
(43) Pub. Date: Mar. 4, 2021

(54) MATERIAL RECOMMENDATION SYSTEM AND MATERIAL RECOMMENDATION METHOD

(71) Applicant: INDUSTRIAL TECHNOLOGY RESEARCH INSTITUTE, Hsinchu (TW)

(72) Inventors: Che-Ming Chang, Hsinchu (TW); Yen-Ting Li, Hsinchu (TW); Kuo-Chan Chiou, Hsinchu (TW); Chun-Wei Su, Hsinchu (TW); Shi-Yun Chern, Hsinchu (TW)

(21) Appl. No.: 17/011,016

(22) Filed: Sep. 3, 2020

Related U.S. Application Data
(60) Provisional application No. 62/895,023, filed on Sep. 3, 2019.

(30) Foreign Application Priority Data
Aug. 11, 2020 (TW) 109127238

(51) Int. Cl. G06N 5/04; G06N 20/00; G06F 16/9035; G06K 9/20; G06K 9/00

(52) U.S. Cl. CPC G06N 5/04; G06N 20/00; G06K 9/00369; G06K 9/2054; G06F 16/9035

(57) ABSTRACT

A material recommendation system and a material recommendation method are provided, which use an analysis module to analyze at least one image to generate reference information, and then a recommendation module receives the reference information to provide target information corresponding to the reference information. By analyzing the image, target information including suitable materials can be quickly provided, thereby greatly accelerating the timeline of product development.

### BACKGROUND

### 1. Technical Field

The present disclosure generally relates to a material recommendation system. More specifically, the present disclosure relates to a material recommendation system and a material recommendation method for selecting suitable materials through artificial intelligence.

### 2. Description of Related Art

New products have been developed every day to improve the quality of life and to facilitate social progress. However, developments of new products not only involve the technical aspects of things, but also the inclusion of suitable materials for manufacturing. At present, in searching for suitable materials, product developers often have to source a different supplier for each individual part, and each part may have different material suppliers depending on its specific requirements. Thus, sourcing materials for all the parts require a considerable amount of time.

Moreover, if customization of a product is needed, for example, in the case of sourcing products for athletes of various kinds of sports, due to the facts that their body shapes and the degree of body extension may be different, the specifications (e.g., stretch rate) required for each part of the body also vary greatly (for example, products for runners focus on leg stretch rate, and products for baseball pitchers focus on arm stretch rate), so the combinations of materials (such as water resistance, flexibility, etc.) required for products (e.g., a smart watch, apparel, etc.) to be used by athletes are very different, which makes it difficult for product developers to find possible combinations of various materials when sourcing materials.

Thus, there is a need in the art to provide a solution that overcomes the aforementioned shortcomings.

### SUMMARY

In order to address the aforementioned shortcomings, a material recommendation system is disclosed, which may include: a host side including an analysis module equipped with a learning mechanism and a recommendation module equipped with a prediction mechanism, the analysis module configured for analyzing at least one image to generate a reference information, and the recommendation module communicatively connected with the analysis module and configured for receiving the reference information and providing a target information corresponding to the reference information; and an operating side communicatively connected with the host side and including a user interface for controlling the host side.

A material recommendation method is further disclosed, which may include the following steps of: analyzing at least one image to generate a reference information by using an analysis module equipped with a learning mechanism; and analyzing the reference information to provide a target information corresponding to the reference information by using a recommendation module equipped with a prediction mechanism.

As can be understood from the above, the material recommendation system and the material recommendation method of the present disclosure is capable of providing target information including suitable materials quickly through image analysis. Thus, compared to the prior art, a product developer can quickly obtain recommendations of material selections using the material recommendation system of the present disclosure to quickly attain the material combination of all the parts, thereby greatly accelerating the timeline of product development.

Moreover, with regard to manufacturing of customized products, such as manufacturing of a smart watch for athletes of a variety kinds of sports, material combinations required for various kinds of athletes can be easily acquired by the product developer using the material recommendation system and the material recommendation method of the present disclosure.

### BRIEF DESCRIPTION OF THE DRAWINGS

FIG. 1A is a schematic diagram depicting the operating architecture of a material recommendation system in accordance with the present disclosure.

FIG. 1B is a flowchart depicting the process of a learning mechanism in FIG. 1A.

FIG. 1C is a flowchart depicting the process of a prediction mechanism in FIG. 1A.

FIG. 1C′ is a flowchart depicting the process of an advanced prediction operation in FIG. 1C.

FIG. 2A is a schematic diagram depicting the functional architecture of a host side of the material recommendation system of the present disclosure.

FIG. 2B is a schematic diagram depicting the process of an analysis module in FIG. 2A.

FIG. 2C is a schematic diagram depicting a machine learning process of the analysis module in FIG. 2A.

FIG. 2D is a schematic diagram showing public data used by the analysis module in FIG. 2A during machine learning.

FIG. 2E is a schematic diagram illustrating known data used by the analysis module in FIG. 2A during machine learning.

FIG. 3A is a flowchart depicting the process of a recommendation module in FIG. 2A.

FIG. 3B is a flowchart depicting a machine learning process of the recommendation module in FIG. 2A.

FIG. 4 is a flowchart illustrating a material recommendation method of the present disclosure.

FIG. 5 is a flowchart illustrating an auxiliary process in FIG. 4.

FIG. 6A is a schematic diagram illustrating the process of using the material recommendation system in recommending materials in accordance with an embodiment of the present disclosure.

FIG. 6B is a schematic diagram illustrating the process of using the material recommendation system in recommending materials in accordance with another embodiment of the present disclosure.

### DETAILED DESCRIPTION

Embodiments of the present disclosure are explained with implementations below. Other advantages and technical effects of the present disclosure can be readily understood by one of ordinary skill in the art upon reading the disclosure provided herein.

It should be noted that the structures, ratios, sizes shown in the drawings appended to this specification are to be construed in conjunction with the disclosure of this specification in order to facilitate understanding of those skilled in the art. They are not meant, in any ways, to limit the implementations of the present disclosure, and therefore have no substantial technical meaning. Without affecting the effects created and objectives achieved by the present disclosure, any modifications, changes or adjustments to the structures, ratio relationships or sizes, are to be construed as fall within the range covered by the technical contents disclosed herein. Meanwhile, terms, such as “first,” “second,” “above,” “below,” “one,” “a,” “an,” and the like, are for illustrative purposes, and are not meant to limit the range implementable by the present disclosure. Any changes or adjustments made to their relative relationships, without modifying the substantial technical contents, are also to be construed as within the range implementable by the present disclosure.

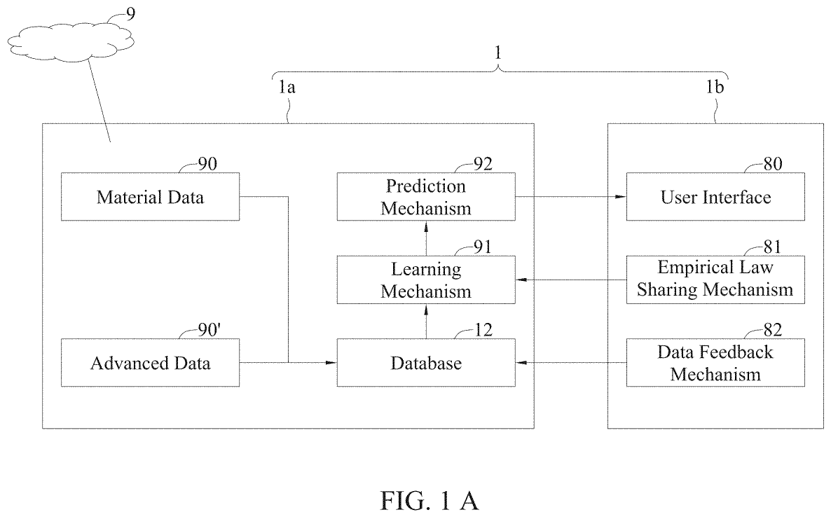

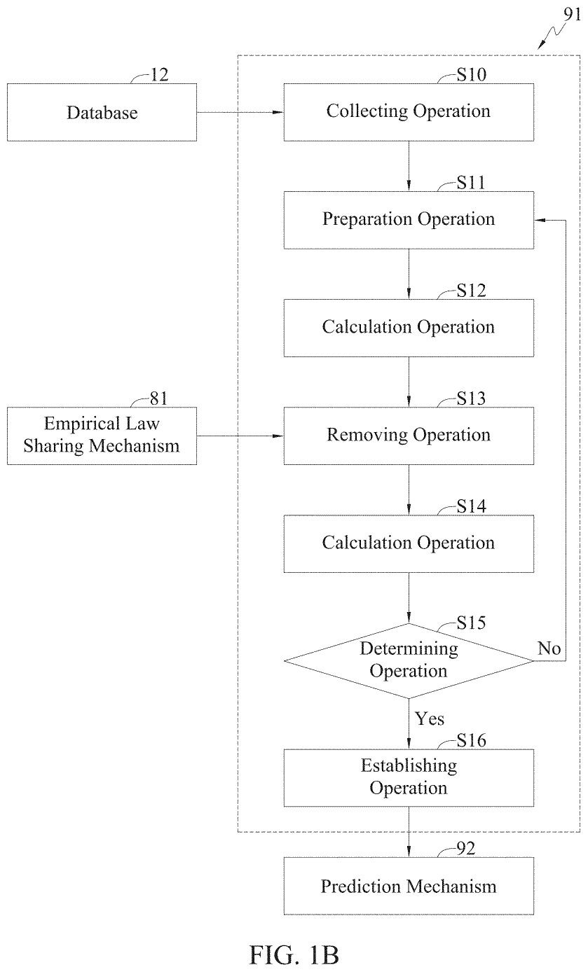

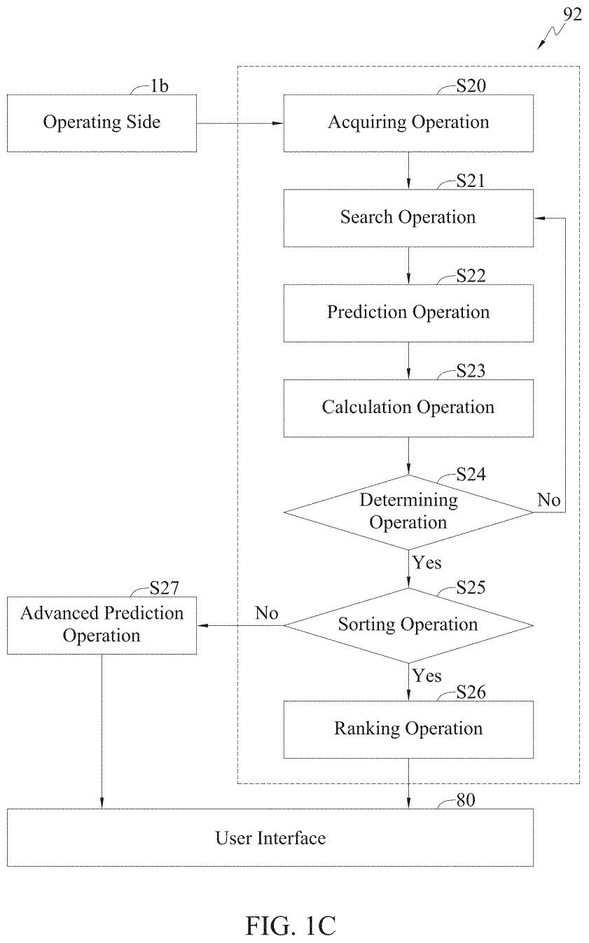

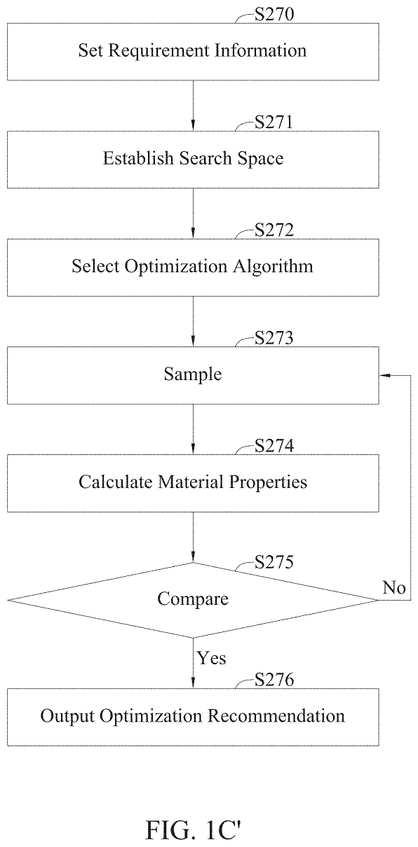

FIG. 1A is a schematic diagram depicting the architecture of a material recommendation system 1 in accordance with the present disclosure. As shown in FIG. 1, the material recommendation system 1 includes a host side 1a and an operating side 1b. The host side 1a is run by an electronic device 9, and the operating side 1b is a client side, which controls the host side 1a via a user interface 80 to obtain target information for manufacturing a target.

In an embodiment, the electronic device 9 is a host computer or a cloud device, which can be communicatively connected (e.g., via network) to different user interfaces 80. The user interface 80 can be configured on, for example, a home computer, a laptop, a smart phone, a tablet or other appropriate 3C (computer, communication and consumer electronics) products.

Moreover, the target is, for example, wearable items, such as clothes or bracelets.

The host side 1a includes a database 12, a learning mechanism 91 and a prediction mechanism 92 (or a predictor 92).

The database 12 can be used for storing material data 90, advanced data 90′ (e.g., testing methods and results thereof, vehicles or other information) or other supplementary data depending on the needs to be used as learning sources for the learning mechanism (i.e., the inputs for the learning mechanism). For example, the material data 90 includes data relevant to various materials, such as flexible materials, soft materials, water-resistant materials, breathable materials, conductive materials or other materials and their properties and sources. Specifically, various datasets can be designed for different types of material according to requirements, such as flexible material dataset, water-resistant material dataset, breathable material dataset, conductive material dataset or other datasets of the specifications required by the target.

The learning mechanism 91 can be an artificial intelligence (AI) training engine. The prediction mechanism 92 can be operated based on the output of the learning mechanism. More specifically, with reference to FIG. 1B, the training process carried out by the learning mechanism 91 is described as below.

In step S10, a collecting operation is performed to receive relevant data from the database 12.

In step S11, a preparation operation is performed to preprocess (e.g., eliminate, classify, format or other actions) the collected data.

In step S12, a calculation operation is performed to process the prepared data using a multicollinearity calculation.

In step S13, a removing operation is performed to remove the limitation of multicollinearity data that has been processed and also data from an empirical law sharing mechanism 81 of the operating side 1b.

In step S14, a calculation operation is performed to learn the removed multicollinearity data using a linear or non-linear algorithm to create new data.

In step S15, a determining operation is performed to determine if the performance of the training conducted by the calculation operation in step S14 is good.

In step S16, if the performance of the training is good, then an establishing operation is performed to establish a recommendation rule for input to the prediction mechanism 92. Otherwise, the process returns to the preparation operation in step S11 for re-learning.

The prediction mechanism 92 is used for performing a prediction operation to recommend (e.g., by way of network transmission) the prediction result to the operating side 1b. More specifically, with reference to FIG. 1C, a prediction process carried out by the prediction mechanism 92 is described as follows.

In step S20, an acquiring operation is performed to receive the recommendation rule from the learning mechanism 91 and requirement information from the operating side 1b, wherein the requirement information is imported to the electronic device 9 from the operating side 1b via the user interface 80, and the requirement information includes vehicles, a target, a score criteria, etc.

In step S21, a search operation is performed to search for all the materials in the database that match the requirement information using a calculation method.

In step S22, a prediction operation is performed to predict combinations of relevant materials for the properties required by the target of the requirement information based on the materials found by the search operation.

In step S23, a calculation operation is performed to calculate a recommendation score for each material based on the various combinations of materials predicted by the prediction operation.

In step S24, a determining operation is performed to determine whether the materials required by the target of the requirement information all need to undergo calculation operation.

In step S25, if the result of step S24 is “yes,” then a sorting operation is performed to select materials that satisfy the score criteria (e.g., the recommendation score is greater than the score criteria) as an accurate combination; else, return to the search operation of step S21 to search again.

In step S26, a ranking operation is performed to rank at least one accurate combination to be used as the prediction result or target information and displayed on the user interface 80 for reference by the operating side 1b, wherein the prediction result or target information includes the material data and their sources. For example, a single flexible material can be obtained from one or more sources, and the sources can be suppliers (e.g., vendors or personnel) or manufacturers (e.g., vendors or personnel).

In step S27, if no accurate combination is generated in step S25, then an advanced prediction (backward predictor) operation (or auxiliary process) is performed. More specifically, with reference to FIG. 1C′, the backward predictor process is described below.

In step S270, properties and vehicles of the target of the requirement information are set.

In step S271, the various properties are classified to establish a plurality of zones to be used as searching space, so as to establish the searching space of the properties.

In step S272, an optimization algorithm is selected, for example, from grid search, random search, Bayesian optimization, Evolutionary algorithm, reinforcement learning, or other suitable algorithms.

In step S273, sampling is performed on each searching space using the optimization algorithm.

In step S274, the material properties of the target are calculated based on the results of the sampling.

In step S275, comparison is performed to determine if there is any material that satisfies the score criteria.

In step S276, if the comparison result is “yes,” then an approximate combination information or optimization recommendation that satisfies the score criteria is created and returned to be displayed on the user interface 80; else, return to step S273 for resampling.

The operating side 1b includes the user interface 80, the empirical law sharing mechanism 81, and a data feedback mechanism 82.

In an embodiment, the user interface 80 is a Graphical User Interface (GUI) to facilitate the operating side 1b in using the material recommendation system 1. Operation options corresponding to the empirical law sharing mechanism 81 and the data feedback mechanism 82 are both configured on the user interface 80.

Furthermore, the prediction mechanism 92 of the host side 1a displays the prediction result or target information on the user interface 80, and the operating side 1b can input relevant materials chose by the users themselves to the learning mechanism 91 at the host side 1a through the empirical law sharing mechanism 81, and the prediction result or target information actually used for manufacturing the target can be supplemented to the database 12 at the host side 1a through the data feedback mechanism 82, thereby improving the performance of the learning mechanism 91, and in turn, the prediction results produced by the prediction mechanism 92.

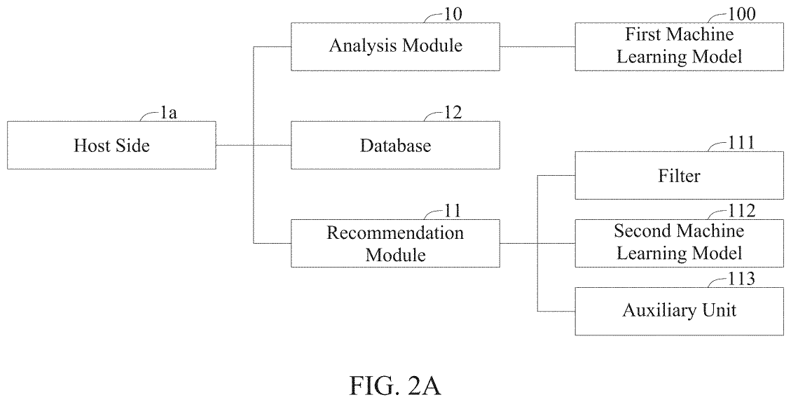

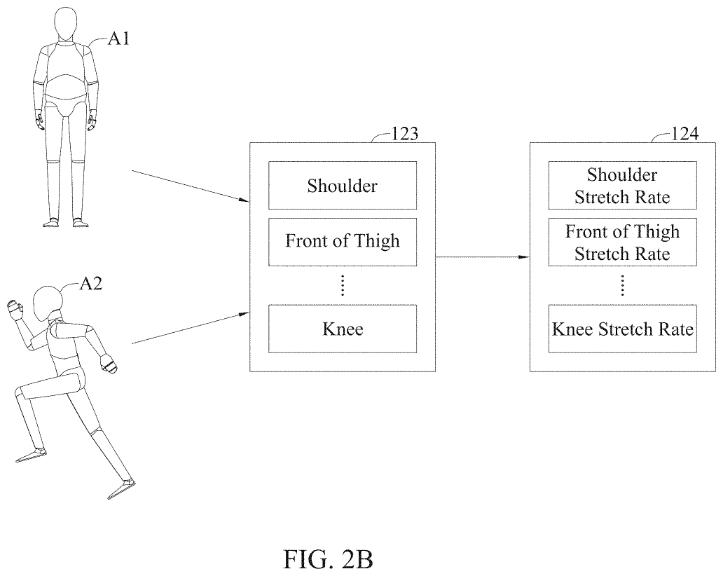

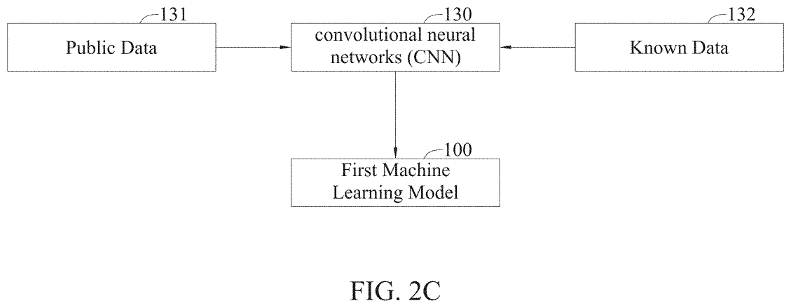

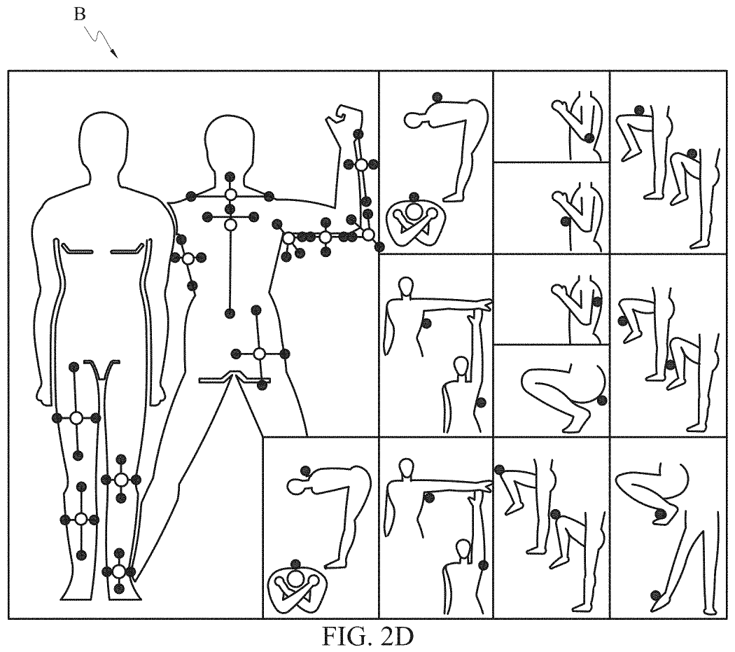

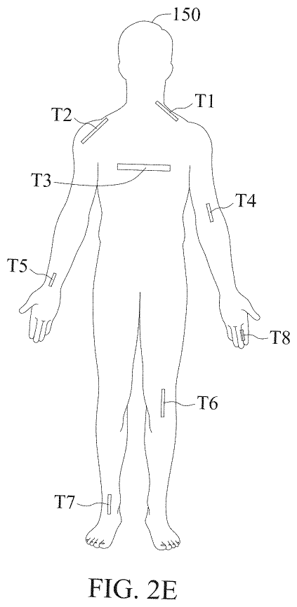

FIG. 2A is a schematic diagram depicting the functional architecture of the host side 1a in the material recommendation system 1 in accordance with the present disclosure. As shown in FIG. 2A, the host side 1a includes: a database 12 shown in FIG. 1A, an analysis module 10 equipped with the learning mechanism 91, and a recommendation module 11 equipped with the prediction mechanism 92.

The analysis module 10 includes an image recognition unit (not shown) for analyzing at least one image to generate reference information based on the recommendation rule formed in step S16 with reference to FIG. 1B.

In an embodiment, the images can be moving images or still images. Preferably, the images at least include different poses in the front and back stages. For example, a single video or a plurality of pictures showing poses of a continuous action.

Moreover, the content of the images include at least a part of the outline of a human body, such as the shoulder, front of the thigh, knees, chest, back, wrists, elbows or other parts of the body. It can be appreciated that the content of the images can include at least a part of other animals or items.

In addition, the reference information includes stretch rate, for example, skin stretch rate of a human body part (with value from 0% to 120%) or mechanical stretch rate of a structure. For example, the analysis module 10 performs prediction on images A1 and A2 to generate a skin stretch rate 124 for a body part 123 of images A1 and A2 (e.g., the shoulder, front of the thigh, knees, chest, back, wrists, elbows or other parts of the body), as shown in FIG. 2B. More specifically, the analysis module 10 includes operating a first machine learning model 100 of the learning mechanism, such as a support vector machine (SVM) model, a convolutional neural networks (CNN) algorithm model, a random forest model, a k-nearest neighbors (KNN) algorithm or other AI models for training (for example, the training process shown in FIG. 2C uses a CNN 130). In addition to using the database 12 for training, the analysis module 10 can also use public data 131 (for example, historical movement images of human or a comparison table B showing average stretch rates of female and male published in Sen'i Kikai Gakkaishi, Journal of the Textile Machinery Society of Japan, 1983, Volume 36, Issue 6, Pages P275-P279 simply shown in FIG. 2D) or other known data 132 (for example, measured stretch rates of various body parts of people of different body shapes making different poses, such as the stretch rates of various body parts of one of the poses shown in FIG. 2E) for training (as inputs into the empirical law sharing mechanism 81 of step S13 shown in FIG. 1B). As such, when a video or picture(s) of a body part of the same person is inputted, the analysis module 10 can predict the skin stretch rate of that body part based on the recommendation rule, wherein the stretch rates of various body parts T1˜T8 of a human body 150 shown in FIG. 2E are exemplified in table 1 below:

TABLE 1

| | Initial Length | Expanded | Stretch | Stretch |
|---|---|---|---|---|
| Part | (cm) | Length (cm) | Value | Rate |
| T1 | 5 | 6 | 1 | 20% |
| T2 | 8 | 10.4 | 2.4 | 30% |
| T3 | 12 | 13 | 1 | 8.3% |
| T4 | 5 | 8 | 3 | 60% |
| T5 | 2 | 2.5 | 0.5 | 25% |
| T6 | 8 | 13 | 5 | 63% |
| T7 | 5 | 6.3 | 1.3 | 26% |
| T8 | 1.5 | 2.4 | 0.9 | 60% |

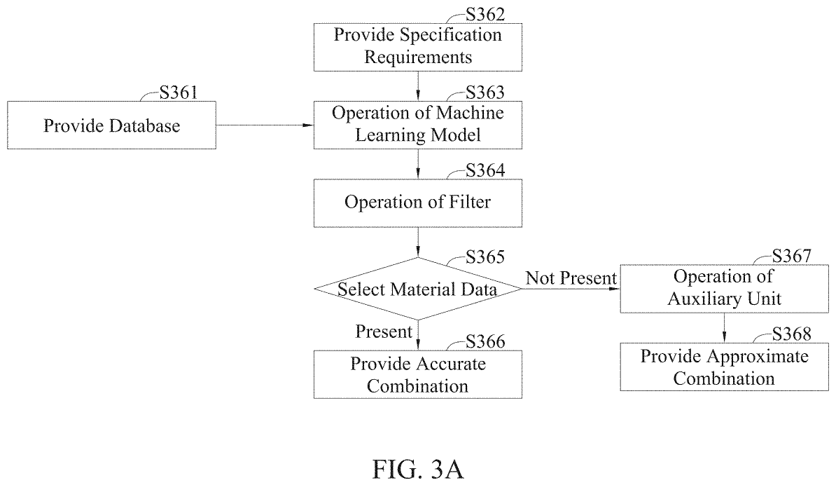

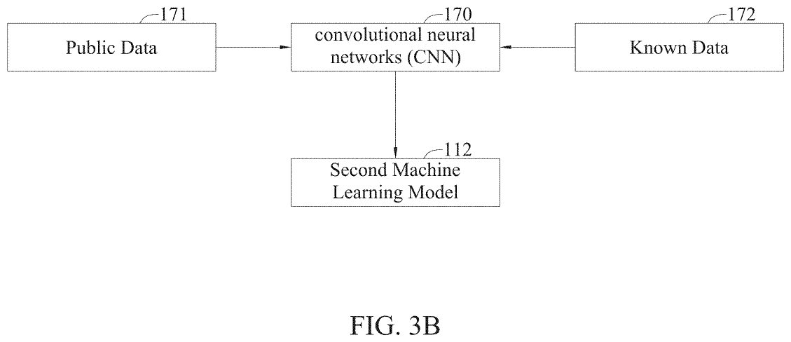

The recommendation module 11 is communicatively connected with the analysis module 10 to receive the reference information, and performs steps S361-S368 of an operation process shown in FIG. 3A, so as to provide the target information (e.g., the prediction result from the ranking operation in step S26) corresponding to the reference information for subsequent manufacturing of the target.

More specifically, in an embodiment, the recommendation module 11 can include a filter 111 in communication with the database 12, so that the filter 111 can select material data of a required target information from the database 12 (e.g., steps S20-S25 with respect to FIG. 1C) to allow the recommendation module 11 to provide the accurate combination of the material data for the requirements of the target (e.g., step S26 with respect to FIG. 1C). As an example, in regards to a baseball pitching movement, as the chest stretch rate is different from the wrist stretch rate, the flexible materials used for these two parts are different, so the filter 111 will select flexible materials required for these two body parts from the database 12 in order to manufacturing an apparel that meets the specifications for stretching.

In addition, in the case that there is not enough material data in the database 12 and the filter 111 cannot find a flexible material that satisfies the stretch specification, the recommendation module 11 can further include an auxiliary unit 113 (which is in communication with the database 12) to determine a flexible material that approximates the stretch specification using algorithms or select a flexible material that approximates the stretch specification from the database 12 (e.g., in step S27 of FIG. 1C and steps S270-S276 of FIG. 1C′). It can be appreciated that the filter 111 and/or the auxiliary unit 113 can also select breathable materials, conductive materials, or materials of other specifications for various body parts from the database 12 in order to manufacture a target that satisfies the specification requirement.

Furthermore, the recommendation module 11 may also include a second machine learning model 112 in communication with the database 12 and the filter 111, which can be, for example, a least absolute shrinkage and selection operator (Lasso) model, a SVM model, a CNN algorithm model, a random forest model, a KNN algorithm or other AI models. As shown in FIG. 3B, the second machine learning model 112 can use public data 171 (e.g., various types of flexible materials and their basic properties) or other known data 172 (various materials and properties actual measured) for training (a CNN 170 is used as an example in the process shown in FIG. 3B), such that when the skin stretch rate or other specification requirements are inputted (at the operating side as described with reference to step S20 in FIG. 1C), the filter 111 can select the required material data through the second machine learning model 112.

In addition, as the sources of the various material data are stored in the database 12, this allows the recommendation module 11 to not only provide an accurate combination or approximate combination of the material data required by the target, the source(s) of each material data can be further provided.

It can be appreciated that if the analysis module 10 uses the first machine learning model 100 for training, then the reference information may include other specification items, such as hardness, and is not limited to stretch rate. Thus, the recommendation module 11 can provide the target information (i.e., an accurate combination or approximate combination of the material data required by the target) for different specification items (e.g., biocompatibility, sweat corrosion resistance, resistance change rate).

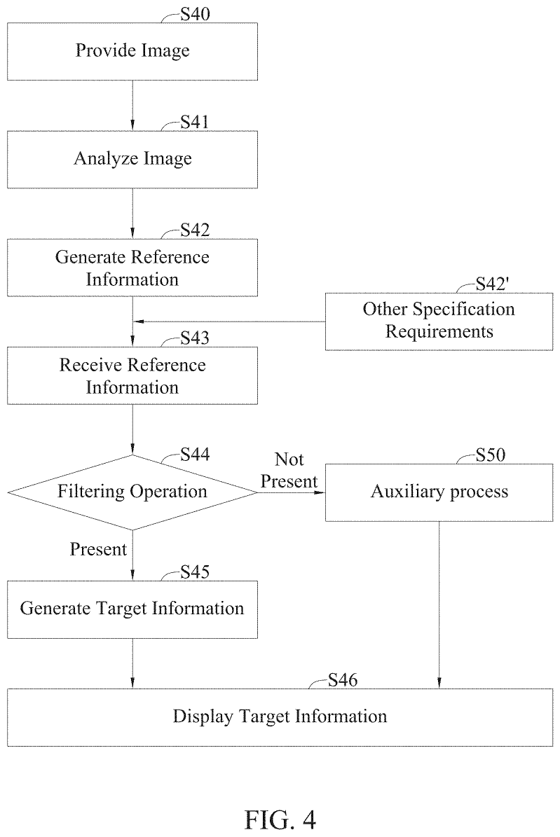

FIG. 4 is a flowchart illustrating a material recommendation method in accordance with present disclosure. As shown in FIG. 4, the material recommendation method is operated in cooperation with the material recommendation system 1. In an embodiment, the target is a sportswear, and the material recommendation method is used for finding the materials needed for the sportswear.

In step S40, at least one image is provided, such as a video or one to five pictures (e.g., photos). In an embodiment, a user can upload the image to the electronic device 9 using the user interface 80.

In step S41, the image is analyzed by the analysis module 10. In an embodiment, the image is inputted to the first machine learning model 100 for image recognition and stretch analysis.

In step S42, reference information is generated by the analysis module 10 based on the recommendation rule. In an embodiment, after image recognition and stretch analysis, the first machine learning model 100 outputs a prediction result, which includes reference information on skin stretch rate (e.g., 20% stretch rate) or other specification requirements.

In step S43, the reference information is inputted into the recommendation module 11 to carry out the prediction mechanism 92. In an embodiment, additional reference information (e.g., water resistance, anti-corrosion, etc.) can be further inputted into the recommendation module 11 using other input methods (e.g., manually inputted at the operating side 1b in step S20 of FIG. 1C), as shown in step S42′, such that the recommendation module 11 receives multiple sets of reference information.

In step S44, filtering is performed by the recommendation module 11 based on these reference information (in steps S21-S25 of FIG. 1C). In an embodiment, these reference information are inputted into the second machine learning model 112, so that after analyzing the reference information, the second machine learning model 112 outputs a prediction result based on the database 12.

In step S45, target information corresponding to the reference information is provided by the recommendation module 11 (in step S26 of FIG. 1C). In an embodiment, if the prediction result shows that there is an accurate combination of material data in the database 12 that satisfies the reference information (for example, a stretch rate of 40%, which is greater than the 20% stretch rate of the reference information, that is, 40%>20%), then the filter 111 can select an accurate combination of material data required from the database 12, and the target information further shows the sources of the material data, for example, the manufacturers (or material suppliers) of the various parts of the sportswear. It should be noted that the accurate combination means that all of the materials satisfy the specification requirement.

In step S46, the target information is displayed on a screen of the user interface 80 for user reference.

On the other hand, in step S50, if the prediction result is unable to find an accurate combination of material data in the database 12 that satisfies the reference information (e.g., there is only a material data with 5% stretch rate in the database 12, which is less than the 20% stretch rate of the reference information, i.e., 5%<20%), then the prediction result is inputted into the auxiliary unit 113 for auxiliary process (or the backward prediction operation described in step S27 of FIG. 1C), such that the auxiliary unit 113 provides an approximate combination of the required material data (e.g., a material with a stretch rate of 18%, which is close to 20% stretch rate in the reference information) as additional target information, which is displayed on the screen of the user interface 80, as shown in step S46. It can be appreciated that the approximate combination means that at least one of the materials does not satisfy the specification requirement.

In an embodiment, the approximate combination of material data can be provided to a material developer for reference in developing a relevant material, and to allow the operating side 1b to supplement the database 12 through the data feedback mechanism 82.

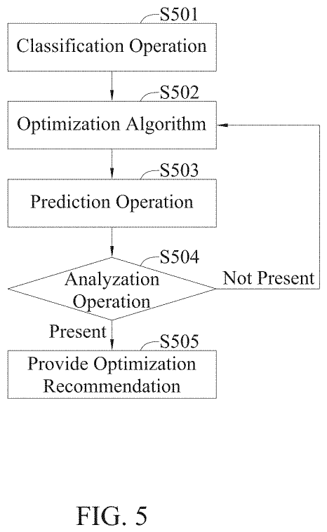

Moreover, the auxiliary process performed by the auxiliary unit 113 is described below with reference to FIG. 5.

In step S501, a classification operation is performed. In an embodiment, the properties of each of the materials are classified to form a plurality of zones to be used as searching space, as described with respect to steps S270-S271. For example, the properties of the materials specified in the reference information are classified into a flexible zone, a water resistance zone, and other zones.

In step S502, an optimization algorithm is performed. In an embodiment, sampling is performed on each searching space using the optimization algorithm, as described with respect to steps S272-S273 of FIG. 1C′. For example, the optimization algorithm includes grid search, random search, Evolutionary algorithm, reinforcement learning, or other suitable algorithms. As such, relevant data, for example, videos, photos, publications or other public data associated with the materials, are selected from the flexible zone, the water resistance zone, and the other zones, respectively.

In step S503, prediction is made based on the results of the sampling. In an embodiment, the stretch rate (or other specifications) is predicted using AI or other algorithms, as described with respect to step S274 of FIG. 1C′.

In step S504, analysis is made on the prediction result outputted by the first machine learning model 100 to determine if there is any material that has the corresponding stretch rate (or other specification), as described with respect to step S275 of FIG. 1C′.

In step S505, if the analysis result indicates that there is a material that has the corresponding stretch rate (or other specification), then an optimization recommendation is provided as the additional target information, as described with respect to step S276 of FIG. 1C′.

On the other hand, if the analysis result indicates that there is no material that has the corresponding stretch rate (or other specification), then the optimization algorithm S502 is performed again.

Therefore, the material recommendation system 1 and the material recommendation method of the present disclosure predicts the skin stretch rate using the analysis module 10 based on images of stretching a body part, and in conjunction with other specification requirement of the target (e.g., a sportswear), obtains an accurate combination of material data using the recommendation module 11. If there is no accurate combination of the material data, then an approximate combination of the material data is proposed as an auxiliary recommendation. In other words, the material recommendation system 1 and the material recommendation method of the present disclosure first conducts searches in the existing data, and if there is no accurate combination of suitable materials in the existing data, it then predicts an approximate combination of the material data using the auxiliary unit 113.

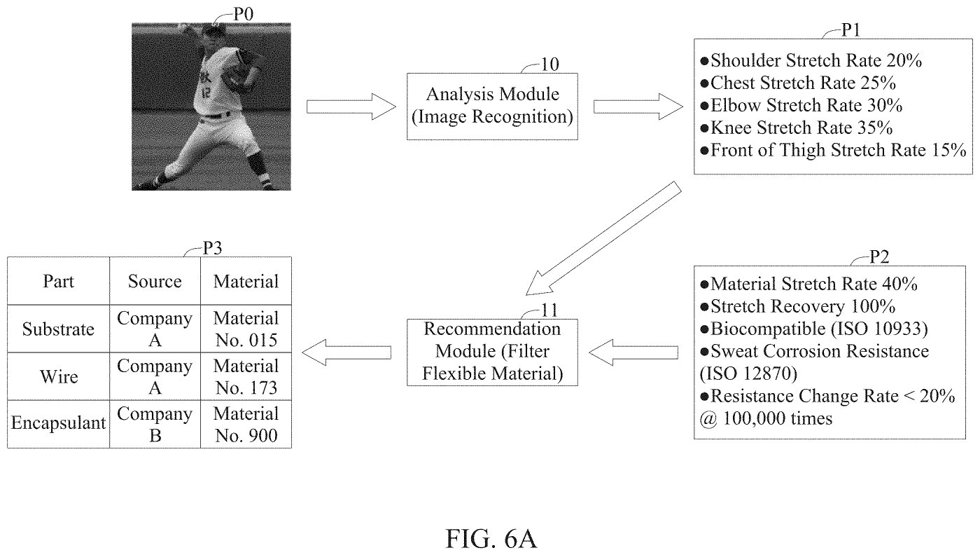

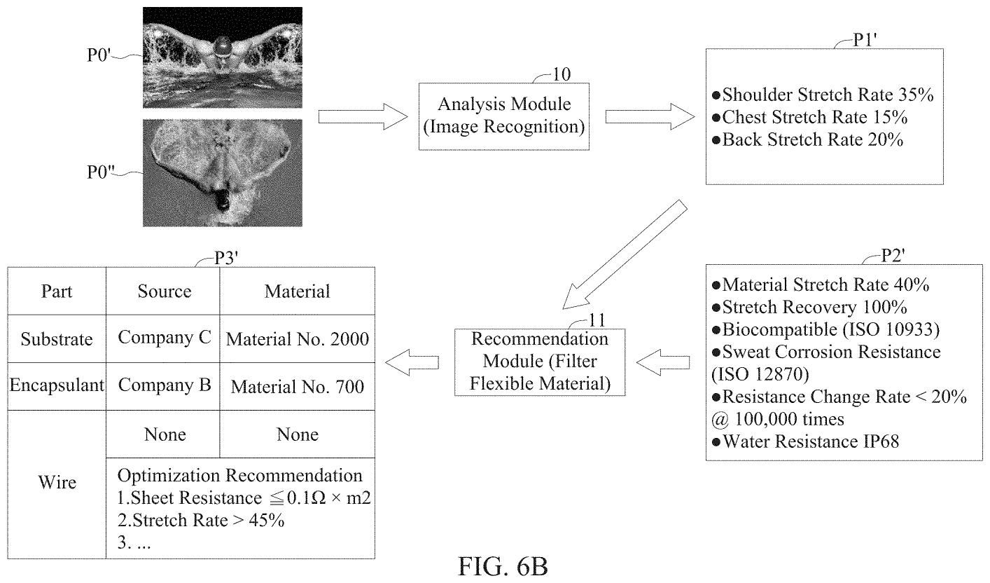

FIG. 6A is a flowchart illustrating actual operations of the material recommendation method using the material recommendation system 1 in accordance with a first embodiment of the present disclosure. In an embodiment, a user enters the host side 1a using the user interface 80 at the operating side 1b to look up materials required for manufacturing a sportswear including an electronic component.

As shown in FIG. 6A, the user uploads an image of a person (e.g., a baseball pitching video P0) to the electronic device 9 via the user interface 80. Then, image recognition is performed by the analysis module 10 to obtain the stretch rates of the various body parts as shown in table P1. Thereafter, reference information associated with the stretch rates are inputted by the analysis module 10 into the recommendation module 11, specifications (e.g., those shown in table P2) required by the sportswear (target) including an electronic component can also be inputted by the user into the recommendation module 11 via the user interface 80. As such, target information associated with the sportswear can be selected by the recommendation module 11, for example, accurate materials (e.g., material No. 15) for various parts (e.g., a substrate, wires, an encapsulant, etc.) of the electronic component and their sources (e.g., company A) can be selected. Finally, the target information (such as table P3) is outputted by the recommendation module 11 to be displayed on the user interface 80 for user reference.

Referring to a second embodiment shown in FIG. 6B, a user uploads images of a person (e.g., swimming photos P0′, P0″) onto the electronic device 9 via the user interface 80. Then, image recognition is performed by the analysis module 10 to obtain the stretch rates of the various body parts as shown in table P1′. Thereafter, reference information associated with the stretch rates are inputted by the analysis module 10 into the recommendation module 11, specifications (e.g., those shown in table P2′) required by the sportswear (target) including an electronic component can also be inputted by the user into the recommendation module 11 via the user interface 80, so that filtering can be performed by the recommendation module 11. When there is no accurate combination of suitable materials within the filtered material range, then the auxiliary process is performed by the auxiliary unit 113 to provide optimization recommendation, for example, a similar material for wires (if there is a material supplier, it will be displayed in the target information. If not, then the user can provide the recommendation to a material supplier for customized development). Finally, the target information (such as table P3′) is outputted by the recommendation module 11 to be displayed on the user interface 80 for user reference.

In summary, the material recommendation system and the material recommendation method of the present disclosure provides target information by image analysis. Therefore, recommendation of material selections can be quickly obtained using the material recommendation system of the present disclosure, greatly reducing the time it takes to source the materials of all the parts, and thus greatly accelerating the timeline of product development.

Moreover, with regards to manufacturing of customized products, material combinations for any customized product can be easily acquired using the material recommendation system of the present disclosure.

While embodiments of the present disclosure have been disclosed in detail herein, it should be appreciated that the present disclosure is not limited thereto or thereby inasmuch as variations on the disclosure herein will be readily appreciated by those of ordinary skill in the art. The scope of the present disclosure shall be appreciated from the claims that follow.

What is claimed is:

1. A material recommendation system, comprising:
a host side including an analysis module equipped with a learning mechanism and a recommendation module equipped with a prediction mechanism, the analysis module configured for analyzing at least one image to generate a reference information, and the recommendation module communicatively connected with the analysis module and configured for receiving the reference information and providing a target information corresponding to the reference information; and
an operating side communicatively connected with the host side and including a user interface for controlling the host side.

2. The material recommendation system of claim 1, wherein the analysis module includes a machine learning model that operates the learning mechanism.

3. The material recommendation system of claim 1, wherein the image includes at least a part of an outline of a human body.

4. The material recommendation system of claim 1, wherein the analysis module analyzes a plurality of the images, and the plurality of the images show poses of a continuous action.

5. The material recommendation system of claim 1, wherein the reference information includes a stretch rate.

6. The material recommendation system of claim 1, wherein the host side further includes a database for storing material data, and the recommendation module is configured to be in communication with a filter of the database, and a material data required is selected from the database by the filter, so as for the target information to include the material data.

7. The material recommendation system of claim 1, wherein the recommendation module includes a machine learning model that operates the prediction mechanism.

8. The material recommendation system of claim 1, wherein the target information includes a material data.

9. The material recommendation system of claim 1, wherein the host side further includes a database for storing material data, and the recommendation module is configured to be in communication with an auxiliary unit of the database, and a material data approximate a required material data is calculated by the auxiliary unit or selected from the database by the auxiliary unit as an additional target information.

10. The material recommendation system of claim 1, wherein the target information includes a source for a material data.

11. A method of material recommendation, comprising:
analyzing at least one image to generate a reference information by using an analysis module equipped with a learning mechanism; and
analyzing the reference information to provide a target information corresponding to the reference information by using a recommendation module equipped with a prediction mechanism.

12. The method of claim 11, wherein the analysis module includes a machine learning model that operates the learning mechanism.

13. The method of claim 11, wherein the image includes at least a part of an outline of a human body.

14. The method of claim 11, wherein the analysis module analyzes a plurality of the images, and the plurality of the images show poses of a continuous action.

15. The method of claim 11, wherein the reference information includes a stretch rate.

16. The method of claim 11, wherein the recommendation module includes a machine learning model that operates the prediction mechanism.

17. The method of claim 11, further comprising storing material data in a database, wherein the recommendation module is configured to be in communication with a filter of the database, and a material data required is selected from the database by the filter, so as for the target information to include the material data.

18. The method of claim 11, wherein the target information includes a material data.

19. The method of claim 11, further comprising storing material data in a database, wherein the recommendation module is configured to be in communication with an auxiliary unit of the database, and a material data approximate a required material data is calculated by the auxiliary unit or selected from the database by the auxiliary unit as an additional target information.

20. The method of claim 11, wherein the target information includes a source for a material data.
